(19) United States
(12) Patent Application Publication
(10) Pub. No.: US 2023/0057930 A1
(43) Pub. Date: Feb. 23, 2023

(54) COMPOSITIONS FOR ALLEVIATING THE SYMPTOMS OF LOW ANTERIOR RESECTION SYNDROME BY TOPICAL ADMINISTRATION TO THE NEORECTUM

(71) Applicant: RepoCeuticals A/S, Hørsholm (DK)

(72) Inventors: Lars Otto Uttenthal, Madrid (ES); Lasse Lindblad, Ålsgårde (DK)

(21) Appl. No.: 17/758,749

(22) PCT Filed: Jan. 14, 2021
(86) PCT No.: PCT/EP2021/050730

§ 371 Date: Jul. 13, 2022

(30) Foreign Application Priority Data
Jan. 16, 2020 (DK) PA202070034

(51) Int. Cl. A61K 31/4045; A61P 1/00; A61K 9/00; A61K 31/10

(52) U.S. Cl. CPC A61K 31/4045; A61P 1/00; A61K 9/0031; A61K 31/10

(57) ABSTRACT

Compositions comprising melatonin and dimethyl sulfoxide are provided for topical administration to the neorectum for use in the alleviation of the symptoms of low anterior resection syndrome.

### FIELD OF INVENTION

The present invention provides compositions comprising melatonin as the essential ingredient for alleviating the symptoms of low anterior resection syndrome by topical administration to the neorectum via the anal canal. As such, it is particularly relevant to the fields of colorectal surgery and gastroenterology.

### BACKGROUND OF THE INVENTION

Low Anterior Resection Syndrome (LARS)

Colorectal cancer (CRC) is the third most commonly diagnosed malignancy and the second leading cause of cancer death worldwide. The World Health Authority (WHO) estimated a total of 1.8 million new cases and almost 861,000 deaths in 2018. The incidence is rising in major parts of the western world, including Europe, and is increasingly affecting people of younger age (<50 years). About 30% of these cases comprise cancer of the rectum.

The standard treatment of rectal cancer is by surgical excision, sometimes preceded by radiotherapy to reduce the risk of cancer recurrence. The standard procedure is to perform a total mesorectal excision. In a minority of cases, a permanent colostomy is created, but in more than ⅔ of the patients, bowel continuity is restored by anastomosis to the anal canal or to a short rectal stump (coloanal or colorectal anastomosis) to preserve the possibility of bowel evacuation via the anus. The anastomosis may be straight, or via a colonic J-pouch, or by side-to-end anastomosis, or via a transverse coloplasty pouch. The anastomosis may be performed at primary surgery or after a period with a defunctioning colostomy which is then reversed. The portion of the colon anastomosed to the anal canal or rectal stump is often referred to as the “neorectum”, a surgically created substitute for the normal rectum.

Symptoms of LARS: Unfortunately, the majority of patients with a coloanal anastomosis do not experience a restoration of normal bowel function. Instead, they experience long-term adverse effects on their quality of life from the symptoms of what has been named “low anterior resection syndrome” or LARS. These comprise frequency, urgency, and/or incontinence of flatus and stool, with diarrhea, or constipation, with a sense of incomplete defecation. These may be associated with a considerable degree of pain and last longer than the initial adaptive postoperative period, persisting for months or years. Patients typically fall into groups with predominance of either diarrhea or constipation with their ancillary symptoms, but some patients have features of both, either almost simultaneously or alternating between the two. The bowel symptom that is most associated with a decreased quality of life has been reported to be diarrhea.

An international consensus definition of low anterior resection syndrome (LARS) has been published by Keane et al (2020; ANZ J Surg 90:300-307), which gives further details of the syndrome herein described and treated. Eight common and characteristic symptoms are listed: variable, unpredictable bowel function; altered stool consistency; increased stool frequency; repeated painful stools; emptying difficulties; urgency; incontinence; and soiling.

Depending on the severity of the symptoms, patients are divided in a group having no LARS (10-15%), minor LARS and major LARS. The latter group may unfortunately constitute over 30% of patients, depending on the center making the assessment.

The deterioration of quality of life in patients with LARS is not just due to the specific abnormalities of bowel movement. Other very significant factors are the associated lower abdominal pain, present between as well as during bowel movements, and serious sleep disturbance resulting in sleep deprivation and chronic fatigue.

Pathophysiology of LARS: The pathophysiology behind the development of LARS is far from being completely understood. Different hypotheses exist as to the contributory causes of LARS, among which a strong one is that the nerve damage in the pelvic region due to radiation therapy and/or surgical dissection of the rectum is of central importance. The nerve damage may result in internal anal sphincter dysfunction, decrease in anal canal sensation, disappearance of the rectoanal inhibitory reflex, and disruption in local reflexes between the anus and the neorectum. Reduction in rectal reservoir capacity and compliance may also be a contributory factor.

After external denervation, contractions of the descending colon are modulated by its intrinsic nerve plexuses, the myenteric and the submucosal plexuses, and the activity of their intrinsic neurons. Serotonin (5-hydroxytryptamine, 5-HT) is an important neurotransmitter in the intrinsic innervation, acting to stimulate motility especially via 5-HT₃ and 5-HT₄ serotonin receptor subtypes. Serotonin receptor antagonists have an alleviating effect on other types of intestinal hypermotility, such as irritable bowel syndrome, and it has been shown that administration of the selective serotonin 5-HT₃ receptor antagonist ramosetron alleviated the symptoms of urgency and reduced the number of bowel movements per day in patients with LARS.

Current treatment options: Different treatment measures have some alleviating effect on the symptoms of LARS. These include bowel irrigation, sacral nerve stimulation via implantable stimulators in patients with medically refractory incontinence, and biofeedback therapy in which patients with bowel outlet dysfunction are trained with myometry and balloons to strain effectively and relax the anal sphincter. These treatments are all invasive, troublesome and time-consuming, and have in general achieved only modest results. In a subgroup of patients, the symptoms are so severe and intractable as to require alleviation by the creation of a permanent colostomy.

Unmet medical need: The preceding considerations clearly demonstrate that there is an unmet medical need for an improved medicinal treatment for the more effective alleviation of the symptoms of LARS. It is the purpose of the present invention to provide such a treatment.

### SUMMARY OF THE INVENTION

The invention consists of providing pharmaceutical compositions comprising melatonin and dimethyl sulfoxide (DMSO) in aqueous solution to alleviate the symptoms of LARS by the direct administration of the compositions via the anal canal to the coloanal anastomosis and the contiguous colonic epithelium (neorectum) in the form of a liquid, foam, or gel. The advantage of the invention is that the melatonin, a modulator of intestinal motility, is delivered at high concentration directly to the colonic region whose motility has to be regulated. The DMSO is not only an excellent solvent for melatonin, which also prevents the decomposition of melatonin in aqueous solution and prolongs the shelf life of the composition, but is also an effective agent to promote the penetration of the melatonin into and through the colonic mucosa to reach the submucosal and myenteric nerve plexuses, where its modulatory action is exerted. Both melatonin and DMSO have analgesic actions to alleviate the pain associated with LARS. In addition, some of the melatonin penetrates into the systemic circulation to reach concentrations that are more than adequate to promote the well-known sleep inducing effect of melatonin in patients with sleep disorders. For this reason, the compositions are administered once daily before the patient retires for the night. It will be seen that that compositions are designed to alleviate the principal symptoms of LARS that adversely affect the patients' quality of life: stool frequency, pain and sleep deprivation.

Accordingly, the basic pharmaceutical composition comprises essentially:

A composition comprising melatonin and DMSO in aqueous solution formulated to be suitable for administration via the anal canal to the neorectum as a liquid, low-viscosity hydrogel, or foam, for use in the alleviation of the symptoms of LARS.

The invention fulfills the unmet medical need for a more effective medicinal treatment to alleviate the symptoms of LARS.

In the following detailed description of the invention, details of the scope of the invention will be given, together with details of the practical performance of the invention.

### DETAILED DESCRIPTION OF THE INVENTION

The present invention provides compositions comprising melatonin and DMSO in aqueous solution to alleviate the symptoms of LARS by the direct administration of the compositions via the anal canal to the coloanal anastomosis and the contiguous colonic epithelium (neorectum) in the form of a liquid, foam, or gel.

Active Ingredients

Melatonin: Melatonin (N-acetyl-5-methoxytryptamine) is an endogenously produced hormone released by the pineal gland, which is involved in regulating circadian bodily rhythms, its release being governed by the suprachiasmatic nucleus. In addition to regulating the sleep/wake cycle, melatonin also has effects on other endogenous hormones such as corticosteroids and is believed to have an effect on the immune system. Melatonin production was first demonstrated in the pineal gland, but there is in fact a much larger production of melatonin by the enterochromaffin cells of the bowel. The role of melatonin in bowel function has not yet been clearly described. Melatonin has been shown to have analgesic effects in addition to its sleep-promoting effect and its effects as a circadian rhythm stabilizer (jetlag and various sleep disorders). Preliminary studies have shown that melatonin has an effect on irritable bowel syndrome with diarrhea as the most prevalent symptom. While low doses of melatonin may increase bowel motility, higher doses may inhibit bowel motility and thus alleviate symptoms of hyperactivity in irritable bowel syndrome. There is melatonin receptor and 5-HT receptor cross-talk in the bowel, and hybridization of melatonin and 5-HT receptor subunits has been described.

A major advantage in using pharmacological doses of melatonin for therapeutic purposes is its extremely low toxicity. The 50% lethal oral dose (LD₅₀ oral) in rats was in excess of 3.2 g/kg, so that no exact figure for the LD₅₀ could be determined. Human subjects have taken gram amounts of oral melatonin per day for prolonged periods without apparent adverse effects.

It will be apparent to the skilled person that the object of the present invention may also be achieved by employing biologically active metabolites, analogues or derivatives of melatonin after verifying that they share the motility-regulating, analgesic and sleep-promoting properties of melatonin itself.

Dimethyl sulfoxide (DMSO): DMSO ((CH₃)₂SO), molecular weight 78.1 g/mol, is a colorless polar aprotic solvent for both polar and nonpolar compounds and is completely miscible with water and a wide range of organic solvents. It is well known as a solvent or solubilizer for melatonin, as are also ethanol, glycerin and propylene glycol. The solubility of melatonin in DMSO at room temperature (20 or 25° C.) is quoted as being as high as a molar concentration (232 g/L) or even more. On the other hand, the solubility of melatonin in water is limited to approximately 2 g/L at 20° C. DMSO shows low toxicity, the median 50% lethal oral dose (LD₅₀ oral) in the rat being twice as high as that of ethanol. DMSO penetrates the skin and other epithelia without damaging them and can carry other compounds dissolved in it into the underlying cells and tissues. DMSO has been used in human subjects as a topical analgesic, a vehicle for the topical application of pharmaceuticals e.g. as a component of a transdermal drug delivery systems, as an anti-inflammatory agent, and as an antioxidant. It is also used as a cryopreservative for sperm and oocytes in fertility treatments as well as for stem cells and modified lymphocytes for cellular therapies for cancer. In the latter circumstances DMSO is routinely given intravenously to patients in doses up to 1 g/kg/day, without evidence of adverse effects. While DMSO has been examined for the treatment of numerous conditions and ailments, its only use approved by the U.S. Food and Drug Administration (FDA) has been for the symptomatic treatment of interstitial cystitis, in which 50% (w/w) aqueous DMSO is instilled into the urinary bladder. The present inventors have determined that DMSO prevents the decomposition of melatonin in aqueous solutions, thus prolonging the shelf life of such preparations, and that the further addition of glycofurol as a solubilizer and penetration enhancer also reduces the decomposition of melatonin.

Formulations

The pharmaceutical composition of the present invention may be in the form of a liquid, low-viscosity gel or foam, which may be delivered by an appropriate mini-enema or foam-containing syringe.

The formulation typically contains from 5 mg to 100 mg of melatonin per gram of the composition.

A preferred formulation consists of 25 mg of melatonin dissolved in 2.5 mL of a 20% w/w glycofurol and 40% w/w DMSO solution in water. This fluid has been demonstrated to show no degradation of melatonin during storage at 25° C. for 45 days at room temperature.

The viscosity of the liquid formulation can be increased, should it be so desired, by the addition limited amounts of pharmaceutically acceptable thickening agents, such as hypromellose or a large number of other pharmaceutically acceptable inert thickening agents known to the skilled person in such formulations.

In this respect, there is a smooth transition between thickened liquids and a low-viscosity hydrogel, which should be no more viscous than to permit delivery via a mini-enema device.

In another embodiment, the formulation is a foam suitable for application to the neorectum. A non-limiting example of such as formulation is a foam based on propylene glycol, comprising additionally the active ingredients emulsifying wax, polyoxyl 10 stearyl ether and cetyl alcohol, all of pharmaceutical grade. The foam also contains conservation agents such as methyl and/or propyl parahydroxybenzoate, lactic acid or triethanolamine as a pH regulator, purified water and a suitable propellant, a non-limiting example of which is a hydrocarbon propellant such as HP-70, containing isobutane and propane.

Many other compositions of foam preparations suitable for application via the anal canal use are known to the skilled person and can be applied to the compositions of this invention.

The volume of the formulation can be varied, from e.g. 2.5 mL to 10 mL depending on the individual circumstances, the concentration of melatonin being adjusted so that its effective dose is maintained, while the concentration of the other ingredients is kept constant.

Administration

Administration of an effective amount of the pharmaceutical composition is by topical application to the neorectal epithelium by means of a mini-enema device or as a foam delivered via a syringe, the application being performed after a bowel movement.

For administration as a foam, the composition may be, as a non-limiting example, provided in a spray can that also contains a propellant. When the nozzle of the can is depressed, a valve opens to allow the composition-propellant mixture to escape, so that a foam is formed by the expansion of the propellant on emerging. The spray can nozzle is applied to the nozzle of an applicator in the form of a syringe to receive the foam. The applicator nozzle is smooth and of sufficient length to deliver the foam to the neorectum. The foam is applied by hand operation of the applicator syringe to ensure adequate control of the volume and rate of delivery. One gram of composition may expand to about 10 mL of foam.

Dose and Dosage Regimens

By “effective amount” of the pharmaceutical compositions of the present invention is meant a dose, which, when administered to a subject in need thereof, achieves a concentration which has a beneficial biological effect, i.e. alleviating the symptoms of LARS. Such an effective amount may be determined by clinicians of ordinary skill in the art attending patients with LARS, by exercising their clinical judgment and/or by reviewing the patient's responses to established bowel-symptom scoring systems, such as the Low Anterior Resection Syndrome Score and the Bowel-Related Quality of Life questionnaire.

The effective amounts and dosages of the ingredients of the composition are not determined in relation to body weight or body surface area, because the treatment is in one aspect topical to the neorectum and subsequent systemic effects to alleviate sleep disturbance have a very wide leeway in the range of achieved blood concentrations.

The effective amount of melatonin for a single dose of topical neorectal administration may be from 5 mg to 100 mg, preferably 10 mg to 90 mg, preferably 20 mg to 75 mg, preferably 25 mg to 50 mg. A preferred amount is 25 mg.

The effective dose is preferably given within an hour before planned retiring at night, such as an hour or less prior to retiring at night.

The composition is used for at least 14 days to establish whether the patient experiences the expected alleviation of symptoms, and if these are alleviated, may be extended for as long as the attending clinician deems necessary.

The composition is preferably administered once a day. It is preferably administered for 14 consecutive days. The composition may be administered for more than 14 consecutive days until alleviation of the symptoms of LARS is achieved.

To establish whether continued treatment is necessary, the attending clinician may decide to interrupt the treatment for a period of a week to a month, and then resume treatment if symptoms recur.

### Example

Clinical Trial to Determine the Effect of a Composition of this Invention Administered Topically to the Neorectum of Patients with LARS

Design: Randomized placebo-controlled crossover trial

Inclusion: Patients with LARS symptoms corresponding to mild or severe LARS within 12 months after the restoration of bowel continuity.

Intervention: Daily administration of the composition containing 25 mg of melatonin by enema at one hour before planned bedtime.

Treatment for 28 days with a 28-day wash out period. Crossover with randomization for placebo (i.e. composition without melatonin and DMSO) or the active medication.

Outcome measures:

Bowel-related quality of life.

LARS score.

Secondary outcome measures include the measurement of cellular changes, inflammatory, immunological and neurological markers in biopsies of the neorectal mucosa, such as transcriptional profiling using the Nanostring immune panel and transcriptional profiling of 5-HT and motilin receptors.

### Embodiments

1. A composition comprising melatonin and dimethyl sulfoxide (DMSO) in an aqueous solution formulated to be suitable for topical administration via the anal canal to the mucosa of the neorectum, as a liquid, gel or foam, for use in the alleviation of the symptoms of low anterior resection syndrome in a patient who has undergone said resection.

2. The composition for use according to embodiment 1, wherein the total single daily dose of melatonin is dissolved in an aqueous medium containing DMSO 40% (w/w).

3. The composition for use according to any of the preceding embodiments, in which the total single daily dose of melatonin is dissolved in an aqueous medium containing DMSO 40% (w/w) and glycofurol 20% (w/w).

4. The composition for use according to any one of the preceding embodiments, wherein the volume of administered composition contains a total single daily dose of 5 mg to 100 mg of melatonin, preferably 25 mg.

5. The composition for use according to any one of the preceding embodiments, wherein the total volume of the administered composition is 2.5 mL to 10 mL of liquid or low-viscosity hydrogel.

6. The composition for use according to embodiments 1 to 4, wherein the composition is administered as a volume of 10 mL of foam.

6. The composition for use according to any one of the preceding claims, wherein a single dose of the composition is administered within one hour before the patient's planned retirement for the night.

7. A method for alleviating the symptoms of low anterior resection syndrome in a patient who has undergone said resection, the method comprising administering a composition comprising melatonin and dimethyl sulfoxide (DMSO) in an aqueous solution formulated to be suitable for topical administration via the anal canal to the mucosa of the neorectum, as a liquid, gel or foam.

8. The method for alleviating the symptoms of low anterior resection syndrome in a patient who has undergone said resection according to the embodiment 7, wherein the total single daily dose of melatonin is dissolved in an aqueous medium containing DMSO 40% (w/w).

9. The method for alleviating the symptoms of low anterior resection syndrome in a patient who has undergone said resection according to any of the embodiments 7 or 8, in which the total single daily dose of melatonin is dissolved in an aqueous medium containing DMSO 40% (w/w) and glycofurol 20% (w/w).

10. The method for alleviating the symptoms of low anterior resection syndrome in a patient who has undergone said resection according to any of the embodiments 7 to 9, wherein the volume of administered composition contains a total single daily dose of 5 mg to 100 mg of melatonin, preferably 25 mg.

11. The method for alleviating the symptoms of low anterior resection syndrome in a patient who has undergone said resection according to any of the embodiments 7 to 10, wherein the total volume of the administered composition is 2.5 mL to 10 mL of liquid or low-viscosity hydrogel.

12. The method for alleviating the symptoms of low anterior resection syndrome in a patient who has undergone said resection according to any of the embodiments 7 to 10, wherein the composition is administered as a volume of 10 mL of foam.

13. The method for alleviating the symptoms of low anterior resection syndrome in a patient who has undergone said resection according to any of the embodiments 7 to 12, wherein a single dose of the composition is administered within one hour before the patient's planned retirement for the night.

1. A method of inhibiting a symptom of low anterior resection syndrome in a patient who has undergone said resection comprising:
topically administering to the mucosa of the neorectum of said patient via the anal canal a composition comprising melatonin and dimethyl sulfoxide (DMSO) formulated in an aqueous solution, as a liquid, gel or foam.

2-6. (canceled)

7. The method according to claim 1, wherein the total single daily dose of melatonin is dissolved in an aqueous medium containing DMSO 40% (w/w) and glycofurol 20% (w/w).

8. The method according to claim 1, wherein the administered composition comprises a total single daily dose of 5 mg to 100 mg of melatonin.

9. The method of claim 1, wherein the administered composition comprises 2.5 mL to 10 mL of liquid or low-viscosity hydrogel.

10. The method of claim 1, wherein the composition is administered as a volume of 10 mL of foam.

11. The method of claim 1, wherein a single dose of the composition is administered within one hour before the patient's planned retirement for the night.
